(19) United States
(12) Patent Application Publication
(10) Pub. No.: US 2021/0139418 A1
(43) Pub. Date: May 13, 2021

(54) PERSONALIZED, ALLOGENEIC CELL THERAPY OF CANCER

(71) Applicant: The Johns Hopkins University, Baltimore, MD (US)

(72) Inventors: Ephraim J. Fuchs, Owings Mills, MD (US); Howard Simons, Toronto (CA)

(21) Appl. No.: 16/953,684

(22) Filed: Nov. 20, 2020

Related U.S. Application Data
(63) Continuation of application No. 15/750,385, filed on Feb. 5, 2018, now abandoned.
(60) Provisional application No. 62/200,385, filed on Aug. 3, 2015.

(51) Int. Cl. C07C 255/54; A61K 35/17; C12N 5/0783; C08G 73/06

(52) U.S. Cl. CPC C07C 255/54; A61K 35/17; C08G 73/0655; C08G 73/0644; C12N 5/0636

(57) ABSTRACT

The present invention describes methods of preparing a cell composition for treating cancer in a human being by obtaining lymphocytes from a partially or fully HLA-matched healthy donor, activating and expanding T cells reactive to neo-antigens (i.e. new epitopes resulting from somatic mutations in the cancer cell), and enriching for tumor-specific T cells that are not reactive against non-tumor tissue of the cancer patient. Provide herein are lymphocyte compositions comprising partially or fully HLA-matched healthy donor T cells reactive to neo-antigens. Also provided herein are methods of treating human cancer with such neo-antigen-specific, non-alloreactive T cells.

### CROSS-REFERENCE TO RELATED APPLICATIONS

This application claims the benefit of priority to U.S. Provisional Application No. 62/200,385, filed Aug. 3, 2015, the entire contents of said application is incorporated herein in its entirety by this reference.

### BACKGROUND

Human cancers typically harbor tens to hundreds of somatic mutations resulting in tumor neo-antigens that can be targeted by self HLA-restricted, tumor-specific T cells. Unfortunately, tumors also can induce functional tolerance in such T cells before they can differentiate into cytotoxic effector cells. T cells from healthy donors are under no such restrictions and, as long as a tumor neo-antigen has been identified, it is possible to generate and expand large numbers of tumor-specific CD4⁺ and CD8⁺ T cells ex vivo under Good

Manufacturing Practice conditions for adoptive immunotherapy in the clinic. A major obstacle to personalized adoptive immunotherapy of cancer has been the difficulty in identifying tumor neo-antigens. As described herein cancer exomes and transcriptomes are sequenced, mutations identified, and HLA-binding algorithms applied to identify novel peptides which bind HLA and are recognized by high affinity, tumor-specific T cells. Substantial advances in graft-versus-host disease prophylaxis and supportive care now make it possible to utilize allogeneic stem cell transplantation (SCT), including partially HLA-mismatched SCT, as a platform for the adoptive immunotherapy of cancer using infusions of ex vivo expanded, tumor-specific T cells from healthy donors.

### SUMMARY OF INVENTION

The present invention describes methods of preparing a cell composition for treating cancer in a human being by obtaining lymphocytes from a partially or fully HLA-matched healthy donor, activating and expanding T cells reactive to tumor neo-antigens (i.e. new epitopes resulting from somatic mutations in the cancer cell), and enriching for tumor-specific T cells that are not reactive against non-tumor tissue of the cancer patient. Provided herein are also methods of treating human cancer with such tumor-specific, non-alloreactive T cells.

Another aspect of the invention is a cellular composition for the treatment of cancer comprising lymphocytes taken from a partially or fully HLA-matched, non-cancerous donor and manipulated so as to contain an increased frequency of tumor-specific T cells and a decreased frequency of alloreactive T cells when compared to the untreated, starting population.

Another aspect of the invention relates to the treatment of patients with metastatic solid cancers comprising the steps of : 1) sequencing the cancer genome and transcriptome from fresh tumor tissue to identify tumor neo-antigens; 2) using amino acid sequence data and HLA-binding algorithms to identify tumor-specific epitopes that bind HLA molecules shared between the patient and a healthy, fully or partially HLA-matched relative; 3) generating and expanding tumor-specific, non-alloreactive CD4⁺ and CD8⁺ T cells from the donor; and 4) treating the patient with reduced intensity allogeneic stem cell transplantation followed by infusion of tumor-specific T cells.

As described herein, the methods and compositions are provided for: 1) identifying of tumor-specific antigens and the use of novel HLA-binding epitopes within these neo-antigens to tailor, or personalize, an allogeneic T cell preparation for the adoptive immunotherapy of cancer; and 2) employing methods to deplete the cell composition of T cells reactive against non-cancerous tissue of the allogeneic recipient. Such methods include physical or chemical methods of selective allodepletion and/or stimulation of T cells with tumor peptides that are not encoded in the genome or transcriptome of non-cancerous cells of the recipient.

One aspect of the invention relates to a method of making an allogeneic lymphocyte composition for treating cancer, the method comprising:

a) providing a peripheral blood composition comprising a population of lymphocytes from a human donor allogeneic to the recipient, said composition comprising T cells, in which the T cells are enriched for T cells reactive to neo-antigens in the recipient and depleted of T cells reactive to antigens on non-cancerous tissues of the recipient,

to thereby generate a population of non-alloreactive T cells depleted of alloreactive T cells.

In some embodiments, the T cell is CD3⁺ T cell.

In some embodiments, the neo-antigens are identified by whole exome sequencing and RNAseq of both cancerous and non-cancerous tissue of the same individual, and HLA binding algorithms applied to determine which neo-antigens bind HLA molecules shared by the donor and recipient.

In some embodiments, the donor has been immunized against one or more neo-antigen of the recipient.

In some embodiments, the immunization consists of intramuscular injection of antigen emulsified in an adjuvant or DNA or mRNA vaccination plus electroporation.

In some embodiments, the T cells from an unvaccinated donor are stimulated ex vivo with one or more neo-antigen, with or without cytokines.

In some embodiments, the one or more neo-antigen used for stimulation is not encoded in the transcriptome or the whole exome of non-cancerous tissue of the same patient.

In some embodiments, the alloreactive T cells are selectively depleted by physical or chemical treatment, or wherein the non-alloreactive T cells are selected for infusion.

Another aspect of the invention relates to an allogeneic lymphocyte composition for administration to a human obtained by any of the methods provided herein for T cell activation, expansion, and isolation.

Another aspect of the invention relates to an allogeneic lymphocyte composition comprising:

a population of T cells reactive to one or more tumor neo-antigens in the recipient, the frequency of such T cells being increased compared to their frequency in a tumor-free donor that has not been vaccinated with one or more neo-antigen.

In some embodiments, the donor is vaccinated with the one or more neo-antigen.

In some embodiments, the T cell is a CD3⁺ T cell.

In some embodiments the lymphocyte composition contains T cells as well natural killer cells and other cells of the peripheral blood

Another aspect of the invention relates to a method of treating a cancer in a human, the method comprising;

(a) administering a lymphocyte composition made by any of the methods provided herein for T cell activation, expansion, and isolation.

In some embodiments, the patient has a clinically, biochemically, or radiographically detectable neoplasm, or has a history of having a neoplasm.

In some embodiments, the lymphocyte composition is administered to a recipient who has undergone an allogeneic stem cell transplantation procedure from the same donor.

In some embodiments, the lymphocyte composition is the first infusion of cells from the donor.

In some embodiments, the lymphocyte composition is infused after treating the recipient with lymphodepleting, but non-myeloablative chemotherapy.

In some embodiments, the lymphocyte composition is infused into a tumor-bearing recipient treated with drugs to augment expression of the at least one antigen recognized by the infused cells.

In some embodiments, the lymphocyte composition is infused into a tumor-bearing recipient in combination with an immunological checkpoint inhibitor.

In some embodiments, the immunological checkpoint inhibitor is selected from ipilimumab, nivolumab, tremelimumab, pidilizumab, or pembrolizumab.

In some embodiments, the lymphocyte composition is infused into a recipient, said recipient having been treated with agents to reduce myeloid-derived suppressor cells or regulatory T cells.

In some embodiments, the lymphocyte composition is infused into a recipient with a neoplasm treated by a local ablative technique.

In some embodiments, the local ablative technique is selected from cryoablation, radiofrequency ablation, high intensity focused ultrasound, irreversible electroporation, external beam radiation, brachytherapy, or chemical destruction.

In some embodiments, the lymphocyte composition is infused with one or more populations of T cells specific for the same neo-antigens or sequentially infused with T cells specific for different neo-antigens.

Another aspect of the invention relates to a method of making an allogeneic lymphocyte composition for treating cancer, the method comprising:

a) providing a peripheral blood composition from a human donor allogeneic to the recipient, the composition comprising T cells, wherein the T cells are enriched for T cells reactive to antigens expressed by the cancer and by the dispensible non-cancerous tissue of the recipient, but not by tissues of the donor, or by other tissues of the recipient with the exception of blood when this infusion is accompanied or preceded by an allogeneic bone marrow transplant from the same donor, and

b) depleting of T cells reactive to antigens on non-cancerous tissues of the recipient.

In some embodiments the dispensible non-cancerous tissue is a sex organ.

Another aspect of the invention relates to a method of making an allogeneic lymphocyte composition for treating cancer, the method comprising:

a) providing a peripheral blood composition from a human donor allogeneic to the recipient, the composition comprising T cells, in which the T cells are enriched for T cells reactive to antigens expressed by the cancer and by the indispensable corresponding non-cancerous tissue of the recipient, but not by tissues of the donor, including the corresponding tissue of the donor that can be transplanted into the recipient, or by other tissues of the recipient, and

b) depleting of T cells reactive to antigens on non-cancerous tissues of the recipient.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

### I. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein the phrase “HLA-binding algorithm” Use of said algorithm provides a means for identifying novel HLA-binding epitopes within neo-antigens to tailor, or personalize, an allogeneic T cell preparation for adoptive immunotherapy of cancer.

The term “immune response” refers herein to any response to an antigen or antigenic determinant by the immune system. Exemplary immune responses include humoral immune responses (e.g. production of antigen-specific antibodies (neutralizing or otherwise)) and cell-mediated immune responses (e.g. lymphocyte proliferation).

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

By the term “neo-antigen,” is meant to refer to novel tumor-specific antigens resulting from somatic mutations in a cancer cell. In some embodiments, cancer exomes and transcriptomes are sequenced, mutations identified, and HLA-binding algorithms applied to identify neo-antigens which bind HLA and are recognized by high affinity, tumor-specific T cells. Novel HLA-binding epitopes within these neo-antigens are used to tailor, or personalize, an allogeneic T cell preparation for the adoptive immunotherapy of cancer

“Activation”, as used herein, refers to the state of a T cell that has been sufficiently stimulated to induce detectable cellular proliferation. Activation can also be associated with induced cytokine production, and detectable effector functions.

The term “activated T cells” refers to, among other things, T cells that are undergoing cell division.

A “stimulatory ligand,” or “stimulatory molecule” as used herein, means a ligand that when present on an antigen presenting cell can specifically bind with a cognate binding partner on a T cell, thereby mediating a primary response by the T cell, including, but not limited to, activation, initiation of an immune response, proliferation, and the like. Stimulatory ligands may comprise HLA-binding epitopes within the neo-antigens of the present invention.

### II. Lymphocyte Compositions

One aspect of the invention provides for a method of making an allogeneic lymphocyte composition for the treatment of cancer in humans, comprising: providing a peripheral blood composition from a human donor allogeneic to the recipient, the composition comprising CD3⁺ T cells, in which the CD3⁺ T cells are enriched for T cells reactive to antigens uniquely expressed by a neoplasm in the recipient and depleted of T cells reactive to antigens on non-cancerous tissues of the recipient.

In some embodiments, the tumor-specific peptides are identified by whole exome sequencing and RNAseq of both cancerous and non-cancerous tissue of the same individual, and HLA binding algorithms applied to determine which tumor-specific peptides bind HLA molecules shared by the donor and recipient

In some embodiments, the donor has been immunized against tumor-specific antigens of the recipient

In some embodiments, the immunization consists of intramuscular injection of antigen emulsified in an adjuvant, DNA vaccination plus electroporation, etc.

In some embodiments, T cells from an unvaccinated donor are stimulated ex vivo with tumor-specific peptides, with or without cytokines

In some embodiments, the amino acid sequence of one or more tumor-specific peptides used for stimulation is not encoded in the transcriptome or the whole exome of non-cancerous tissue of the same patient

In some embodiments, the alloreactive T cells are selectively depleted by physical or chemical treatment, or in which non-alloreactive T cells are selected for infusion.

Another aspect of the invention relates an allogeneic lymphocyte composition for administration to a human recipient obtained by the methods set forth above. Another aspect of the invention relates to an allogeneic human lymphocyte composition comprising:

CD3+ cells containing T cells reactive to one or more tumor neoantigens in the recipient, the frequency of such T cells being increased compared to their frequency in a tumor-free donor that has not been vaccinated with the tumor neoantigen(s), with or without natural killer cells and other cells of the peripheral blood.

Another aspect of the invention relates to a method of making an allogeneic lymphocyte composition for the treatment of cancer in humans, comprising: providing a peripheral blood composition from a human donor allogeneic to the recipient, the composition comprising CD3+ T cells, in which the CD3+ T cells are enriched for T cells reactive to antigens expressed by the cancer and by the dispensible (e.g. sex organs) non-cancerous tissue of the recipient but not by tissues of the donor or by other tissues of the recipient with the exception of blood when this infusion is accompanied or preceded by an allogeneic bone marrow transplant from the same donor, and depleted of T cells reactive to antigens on non-cancerous tissues of the recipient.

Another aspect of the invention relates to a method of making an allogeneic lymphocyte composition for the treatment of cancer in humans, comprising: providing a peripheral blood composition from a human donor allogeneic to the recipient, the composition comprising CD3+ T cells, in which the CD3+ T cells are enriched for T cells reactive to antigens expressed by the cancer and by the indispensable corresponding non-cancerous tissue of the recipient, but not by tissues of the donor, including the corresponding tissue of the donor that can be transplanted into the recipient, or by other tissues of the recipient, and depleted of T cells reactive to antigens on non-cancerous tissues of the recipient.

a. Sources of T cells

Prior to expansion, a source of T cells is obtained from a subject. The term “subject” is intended to include living organisms in which an immune response can be elicited (e.g., mammals). Examples of subjects include humans, dogs, cats, mice, rats, and transgenic species thereof. In some embodiments, the subject is a partially or fully HLA-matched healthy donor (i.e., non-cancerous donor). T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as ficoll separation. In one embodiment of the invention, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in some embodiment, T cells are isolated by incubation with anti-CD3/anti-CD28 (i.e., 3×28)-conjugated beads, such as DYNABEADS™, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells. Further, use of longer incubation times can increase the efficiency of capture of T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, or from samples where there are many tumor cells present (i.e., leukemic blood, tumor tissue, etc.). Such populations of cells may have therapeutic value and would be desirable to obtain.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

If desired or necessary, T cell populations (i.e., CD3⁺ cells) may be depleted from blood preparations prior to ex vivo expansion by a variety of methodologies, including anti-CD3 coated beads or columns, or utilization of the phagocytotic activity of these cells to facilitate removal, or by the use of counterflow centrifugal elutriation. Accordingly, in one embodiment, the invention uses paramagnetic particles of a size sufficient to be engulfed by phagocytotic monocytes. In certain embodiments, the paramagnetic particles are commercially available beads, for example, those produced by Dynal AS under the trade name Dynabeads™. Exemplary Dynabeads™ in this regard are M-280, M-450, and M-500. In one aspect, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments the irrelevant beads include beads coated with sheep anti-mouse antibodies, goat anti-mouse antibodies, and human serum albumin.

In brief, such depletion of monocytes is performed by preincubating PBMC isolated from whole blood or apheresed peripheral blood with one or more varieties of irrelevant or non-antibody coupled paramagnetic particles at any amount that allows for removal of monocytes (approximately a 20:1 bead:cell ratio) for about 30 minutes to 2 hours at 22 to 37° C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL™ Magnetic Particle Concentrator (DYNAL MPC™)). Assurance of requisite depletion can be monitored by a variety of methodologies known to those of ordinary skill in the art, including flow cytometric analysis of CD14 positive cells, before and after said depletion.

T cells for stimulation can also be frozen after the washing step, which does not require the monocyte-removal step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

In some embodiments, the lymphocytes are taken from a partially or fully HLA-matched, non-cancerous donor.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo, Likewise, following ex vivo manipulation using the methods described herein, these cells may be in a preferred state for enhanced engraftment and in vivo expansion. Thus, it is contemplated within the context of the present invention to collect blood cells, including T cells or other cells of the hematopoetic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

b. Activation, Expansion, and Isolation of T Cells

T cells are activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 7,572,631.

One aspect of the invention relates to a method of making an allogeneic lymphocyte composition for the treatment of cancer in humans, comprising: providing a peripheral blood composition from a human donor allogeneic to the recipient, the composition comprising CD3+ T cells, in which the CD3+ T cells are enriched for T cells reactive to antigens uniquely expressed by a neoplasm in the recipient and depleted of T cells reactive to antigens on non-cancerous tissues of the recipient

Another aspect of the invention a method of making an allogeneic lymphocyte composition for the treatment of cancer in humans, comprising: providing a peripheral blood composition from a human donor allogeneic to the recipient, the composition comprising CD3+ T cells, in which the CD3+ T cells are enriched for T cells reactive to antigens expressed by the cancer and by the dispensible (e.g. sex organs) non-cancerous tissue of the recipient but not by tissues of the donor or by other tissues of the recipient with the exception of blood when this infusion is accompanied or preceded by an allogeneic bone marrow transplant from the same donor, and depleted of T cells reactive to antigens on non-cancerous tissues of the recipient.

Another aspect of the invention relates to a method of making an allogeneic lymphocyte composition for the treatment of cancer in humans, comprising: providing a peripheral blood composition from a human donor allogeneic to the recipient, the composition comprising CD3+ T cells, in which the CD3+ T cells are enriched for T cells reactive to antigens expressed by the cancer and by the indispensable corresponding non-cancerous tissue of the recipient, but not by tissues of the donor, including the corresponding tissue of the donor that can be transplanted into the recipient, or by other tissues of the recipient, and depleted of T cells reactive to antigens on non-cancerous tissues of the recipient.

From said method, lymophocyte populations are generated for administration to a human recipient. In certain embodiments, the allogeneic human lymphocyte composition comprising:

CD3⁺ cells containing T cells reactive to one or more tumor neoantigens in the recipient, the frequency of such T cells being increased compared to their frequency in a tumor-free donor that has not been vaccinated with the tumor neoantigen(s), with or without natural killer cells and other cells of the peripheral blood.
III. Methods of treatment

One aspect of the invention relates to a method of treating a neoplasm in a human subject, comprising;

administering a lymphocyte composition made by the methods set forth in section II above.

In some embodiments, the patient has a clinically, biochemically, or radiographically detectable neoplasm, or has a history of having a neoplasm.

In some embodiments, the lymphocyte composition is administered to a recipient who has undergone an allogeneic stem cell transplantation procedure from the same donor.

In some embodiments, the lymphocyte composition is the first infusion of cells from the donor.

In some embodiments, the lymphocyte composition is infused after treating the recipient with lymphodepleting but non-myeloablative chemotherapy.

In some embodiments, the lymphocyte composition is infused into a tumor-bearing recipient treated with drugs to augment expression of the antigen(s) recognized by the infused cells.

In some embodiments, the lymphocyte is infused into a tumor-bearing recipient also treated with an immunologic checkpoint inhibitor such as ipilimumab, nivolumab, or pembrolizumab.

In some embodiments, the lymphocyte composition is infused into a recipient also treated with agents to reduce myeloid-derived suppressor cells and/or regulatory T cells.

In some embodiments, the lymphocyte composition is infused into a recipient with a neoplasm treated by a local ablative technique such as cryoablation, radiofrequency ablation, high intensity focused ultrasound, irreversible electroporation, external beam radiation, brachytherapy, chemical destruction.

In some embodiments, the method of administering neoantigen-specific T cells in one or more infusions containing the T cells specific for the same neoantigens or sequential infusions of T cells specific for different neoantigens.

One aspect of the invention relates to methods of treating human cancers with the tumor-specific, non-alloreactive T cells. The cancer includes but is not limited to brain cancers (e.g., gliomas), lung cancers, liver cancers, cervical cancers, soft tissue sarcomas, endocrine tumors, hematopoietic cancers, melanomas, bladder cancers, breast cancers, pancreatic cancers, prostate cancers, colon cancers, and ovarian cancers. The cancer also can be characterized as benign or malignant. In one embodiment, the cancer is a high grade glioma. In another embodiment, the high grade glioma is a glioblastoma multiforme, an anaplastic astrocytoma, or an oligodendroglioma. Another aspect of the invention relate to a method of treating a neoplasm in a human subject, comprising administering a lymphocyte composition made by the methods set forth in IIb above.

Infusion of the lymphocyte population of the present invention enhances, potentiates, or increases the human's immune response. Generally, the immune response can include the humoral immune response, the cell-mediated immune response, or both. For example, antigen presentation through an immunological pathway involving MHC II proteins or direct B-cell stimulation can produce a humoral response; and, antigens presented through a pathway involving MHC I proteins can elicit the cellular arm of the immune system.

A humoral response can be determined by a standard immunoassay for antibody levels in a serum sample from the subject receiving the pharmaceutically acceptable composition. A cellular immune response is a response that involves T cells and can be determined in vitro or in vivo. For example, a general cellular immune response can be determined as the T cell proliferative activity in cells (e.g., peripheral blood leukocytes (PBLs)) sampled from the subject at a suitable time following the administering of a pharmaceutically acceptable composition. Following incubation of e.g., PBMCs with a stimulator for an appropriate period, [³H]thymidine incorporation can be determined. The subset of T cells that is proliferating can be determined using flow cytometry. T cell cytotoxicity (CTh) can also be determined.

In one embodiment, the immune response that is elicited is sufficient for prophylactic or therapeutic treatment of a neoplastic disease, or a symptom associated therewith. Accordingly, a beneficial effect of the pharmaceutically acceptable composition will generally at least in part be immune-mediated, although an immune response need not be positively demonstrated in order for the compositions and methods described herein to fall within the scope of the present invention.

Administering to both human and non-human vertebrates is contemplated within the scope of the present invention. Veterinary applications also are contemplated. Generally, the subject is any living organism in which an immune response can be elicited. Examples of subjects include, without limitation, humans, livestock, dogs, cats, mice, rats, and transgenic species thereof.

The subject can either have a neoplastic disease (e.g., a tumor), or be at risk of developing the neoplastic disease. Subjects can be characterized by clinical criteria, for example, those with advanced neoplastic disease or high tumor burden exhibiting a clinically measurable tumor. A clinically measurable tumor is one that can be detected on the basis of tumor mass (e.g., by palpation, MRI, CAT scan, X-ray). Thus, for example, the pharmaceutically acceptable composition in accordance with the present invention can be administered to subjects with advanced disease with the objective of mitigating their condition. Preferably, a reduction in tumor mass occurs as a result of administering the pharmaceutically acceptable composition of the present invention, but any clinical improvement constitutes a benefit. Clinical improvement includes decreased risk or rate of progression or reduction in pathological consequences of a tumor, for example.

By way of another example, the subject can be one that has a history of cancer and has been responsive to another mode of therapy. The other therapy may have included e.g., surgical resection, radiotherapy, chemotherapy, and other modes of immunotherapy whereby as a result of the other therapy, the subject presents no clinically measurable tumor. However, the subject can be one determined to be at risk for recurrence or progression of the cancer, either near the original tumor site, or by metastases. Such subjects can be further categorized as high-risk and low-risk subjects. The subdivision can be made on the basis of features observed before or after the initial treatment. These features are known in the clinical arts, and are suitably defined for each different cancer. Features typical of high risk subgroups are those in which the tumor has invaded neighboring tissues, or which show involvement of lymph nodes. Thus, for example, a pharmaceutically acceptable composition of the present invention can be administered to the subject to elicit an anti-cancer response primarily as a prophylactic measure against recurrence. Preferably, administering the composition delays recurrence of the cancer, or more preferably, reduces the risk of recurrence (i.e., improves the cure rate). Such parameters can be determined in comparison with other patient populations and other modes of therapy.

The pharmaceutically acceptable composition can be administered at any time that is appropriate. For example, the administering can be conducted before or during traditional therapy of a subject having a tumor burden, and continued after the tumor becomes clinically undetectable. The administering also can be continued in a subject showing signs of recurrence.

The pharmaceutically acceptable composition can be administered in a therapeutically or a prophylactically effective amount, wherein the pharmaceutically acceptable composition comprises the lymphocyte population of T cells are enriched for T cells reactive to neo-antigens in the recipient and depleted of T cells reactive to antigens on non-cancerous tissues of the recipient, either alone or in combination with one or more other antigens. Administering the pharmaceutically acceptable composition of the present invention to the subject can be carried out using known procedures, and at dosages and for periods of time sufficient to achieve a desired effect. For example, a therapeutically or prophylactically effective amount of the pharmaceutically acceptable composition, can vary according to factors such as the age, sex, and weight of the subject. Dosage regima can be adjusted by one of ordinary skill in the art to elicit the desired immune response including immune responses that provide therapeutic or prophylactic effects.

The pharmaceutically acceptable composition can be administered to the subject at any suitable site, for example a site that is distal to or proximal to a primary tumor. The route of administering can be parenteral, intramuscular, subcutaneous, intradermal, intraperitoneal, intranasal, intravenous (including via an indwelling catheter), via an afferent lymph vessel, or by any other route suitable in view of the neoplastic disease being treated and the subject's condition. Preferably, the dose will be administered in an amount and for a period of time effective in bringing about a desired response, be it eliciting the immune response or the prophylactic or therapeutic treatment of the neoplastic disease and/or symptoms associated therewith.

The pharmaceutically acceptable composition can be given subsequent to, preceding, or contemporaneously with other therapies including therapies that also elicit an immune response in the subject. For example, the subject may previously or concurrently be treated by chemotherapy (e.g., by an alkylating agent such as temozolomide), radiation therapy, and other forms of immunotherapy, such other therapies preferably provided in such a way so as not to interfere with the immunogenicity of the compositions of the present invention.

Administering can be properly timed by the care giver (e.g., physician, veterinarian), and can depend on the clinical condition of the subject, the objectives of administering, and/or other therapies also being contemplated or administered. In some embodiments, an initial dose can be administered, and the subject monitored for either an immunological or clinical response, preferably both. Suitable means of immunological monitoring include using patient's peripheral blood lymphocyte (PBL) as responders and neoplastic cells as stimulators. An immunological reaction also can be determined by a delayed inflammatory response at the site of administering. One or more doses subsequent to the initial dose can be given as appropriate, typically on a monthly, semimonthly, or preferably a weekly basis, until the desired effect is achieved. Thereafter, additional booster or maintenance doses can be given as required, particularly when the immunological or clinical benefit appears to subside.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the lymphocyte composition of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Generally, the cells activated and expanded as described herein may be utilized in the treatment and prevention of diseases that arise in individuals who are immunocompromised. In particular, the lymphocyte compositions of the invention are used in the treatment of cancer. In certain embodiments, the lymphocyte compositions of the invention are used in the treatment of patients at risk for developing cancer. Thus, the present invention provides methods for the treatment or prevention of cancer comprising administering to a subject in need thereof, a therapeutically effective amount of the lymphocyte compositions of the invention.

The lymphocyte compositions of the present invention may be administered either alone, or as a pharmaceutical composition in combination with diluents and/or with other components such as IL-2 or other cytokines or cell populations. Briefly, pharmaceutical lymphocyte compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the lymphocytes described herein may be administered at a dosage of 10⁴ to 10⁹ cells/kg body weight, preferably 10⁵ to 10⁶ cells/kg body weight, including all integer values within those ranges. Lymphocyte compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the lymphocyte compositions of the present invention are administered to a patient by intradermal or subcutaneous injection. In another embodiment, the lymphocyte compositions of the present invention are preferably administered by i.v. injection. The lymphocyte compositions may be injected directly into a tumor, lymph node, or site of infection.

The dosage for treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

Substantial advances in graft-versus-host disease prophylaxis and supportive care now make it possible to utilize allogeneic stem cell transplantation (SCT), including partially HLA-mismatched SCT, as a platform for the adoptive immunotherapy of cancer using infusions of ex vivo expanded, tumor-specific T cells from healthy donors.

Accordingly, in one aspect, the invention provides a method for an allogeneic lymphocyte composition for treating cancer, the method comprising:

a) providing a peripheral blood composition from a human donor allogeneic to the recipient, said composition comprising CD3⁺ T cells, in which the CD3⁺ T cells are enriched for T cells reactive to antigens uniquely expressed by a neoplasm in the recipient and depleted of T cells reactive to antigens on non-cancerous tissues of the recipient.

In some embodiments, the invention provides a method for treatment or prophylaxis of a neoplastic disease or symptoms associated with cancer, the method comprising administering to the subject an effective amount of the lymphocytes described above. In one embodiment, the lymphocytes are administered systemically, preferably by injection. Alternately, one can administer locally rather than systemically, for example, via injection directly into tissue, preferably in a depot or sustained release formulation. Furthermore, one can administer in a targeted drug delivery system, for example, in a liposome that is coated with tissue-specific antibody. The liposomes can be targeted to and taken up selectively by the tissue. In another embodiment, the invention provides use of the lymphocytes in the preparation of a medicament for eliciting an immune response to a cell that expresses a tumor specific antigen, preferably for treating or preventing cancer.

The lymphocytes described above can be administered to a subject, either by themselves or in combination, for eliciting an immune response, particularly for eliciting an immune response to cells that express a neo-antigen. Such cell-based compositions are useful, therefore, for treating or preventing cancer. The cells can be introduced into a subject by any mode that elicits the desired immune response to cells that express a neo-antigen. Furthermore, the lymphocytes can be derived from the subject (i.e., autologous cells) or from a different subject that is MHC matched or mismatched with the subject (e.g., allogeneic). The injection site can be selected from subcutaneous, intraperitoneal, intramuscular, intradermal, intravenous, or intralymphoid.

Single or multiple administrations of the lymphocytes can be carried out with cell numbers and treatment being selected by the care provider (e.g., physician). Preferably, the lymphocytes are administered in a pharmaceutically acceptable carrier. Suitable carriers can be the growth medium in which the cells were grown, or any suitable buffering medium such as phosphate buffered saline. The cells can be administered alone or as an adjunct therapy in conjunction with other therapeutics.

Accordingly, the invention contemplates methods for treatment and/or prophylaxis of a cell that expresses a tumor specific antigen, the method comprising administering to a subject in need of such treatment or prophylaxis a therapeutically/prophylactically effective amount of a lymphocyte composition as described herein.

Techniques for formulating and administering can be found in Remington's Pharmaceutical Sciences, Mack Publishing Co., Easton, Pa., latest edition. Suitable routes can, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic/prophylactic compositions of the present invention can be formulated in aqueous solutions, preferably in physiologically compatible buffers such as Hanks' solution, Ringer's solution, or physiological saline buffer.

The immune response induced in a subject by administering T cells activated and expanded using the methods described herein, or other methods known in the art wherein T cells are stimulated and expanded to therapeutic levels, may include cellular immune responses mediated by cytotoxic T cells, capable of killing tumor and infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present invention, which are well described in the art; e.g., Coligan et al. Current Protocols in Immunology, John Wiley & Sons Inc. (1994).

Typically, in adoptive immunotherapy studies, T cells are administered approximately at 2×10⁹ to 2×10¹¹ cells to the patient. (See, e.g., U.S. Pat. No. 5,057,423). In some aspects of the present invention, particularly in the use of allogeneic or xenogeneic cells, lower numbers of cells, in the range of 10⁶/kilogram (10⁶-10¹¹ per patient) may be administered. In certain embodiments, T cells are administered at 1×10⁵, 1×10⁶, 1×10⁷, 1×10⁸, 1×10⁹, 1×10¹⁰, 1×10¹¹, or 1×10¹², cells to the subject. T cell compositions may be administered multiple times at dosages within these ranges. The cells may be autologous to the patient undergoing therapy. If desired, the treatment may also include administration of mitogens (e.g., PHA) or lymphokines, cytokines, and/or chemokines (e.g., GM-CSF, IL-4, IL-7, IL-13, Flt3-L, RANTES, MIP1α, etc.) as described herein to enhance induction of the immune response.

In certain embodiments, it may be desired to administer activated T cells to a subject and then subsequently redraw blood (or have a leukapheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol, may select out certain populations of T cells.

In yet another embodiment, the pharmaceutical composition can be delivered in a controlled release system. In one embodiment, a pump may be used (see Langer, 1990, Science 249:1527-1533; Sefton 1987, CRC Crit. Ref. Biomed. Eng. 14:201; Buchwald et al., 1980; Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983; J. Macromol. Sci, Rev. Macromol. Chem.

23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, thus requiring only a fraction of the systemic dose (see, e.g., Medical Applications of Controlled Release, 1984, Langer and Wise (eds.), CRC Pres., Boca Raton, Fla., vol. 2, pp. 115-138).

The T cell compositions of the present invention may also be administered using any number of matrices. Matrices have been utilized for a number of years within the context of tissue engineering (see, e.g., Principles of Tissue Engineering (Lanza, Langer, and Chick (eds.)), 1997. The present invention utilizes such matrices within the novel context of acting as an artificial lymphoid organ to support, maintain, or modulate the immune system, typically through modulation of T cells. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. Nos. 5,980,889; 5,913,998; 5,902,745; 5,843,069; 5,787,900; or 5,626,561 are utilized. Matrices comprise features commonly associated with being biocompatible when administered to a mammalian host. Matrices may be formed from both natural or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for cancer patients. In further embodiments, the T cells of the invention may be used in combination with chemotherapy, radiation, irrimunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993; Isoniemi (supra)). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

### INCORPORATION BY REFERENCE

The contents of all references, patent applications, patents, and published patent applications, as well as the Figures and the Sequence Listing, cited throughout this application are hereby incorporated by reference in their entirety as if each individual publication or patent was specifically and individually incorporated by reference. In case of conflict, the present application, including any definitions herein, may control.

### EQUIVALENTS

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. While specific embodiments of the subject invention have been discussed, the above specification is illustrative and not restrictive.

Many variations of the invention may become apparent to those skilled in the art upon review of this specification. The full scope of the invention should be determined by reference to the claims, along with their full scope of equivalents, and the specification, along with such variations. Such equivalents are intended to be encompassed by the following claims.

What is claimed:

1. A method of making an allogeneic lymphocyte composition for treating cancer, the method comprising:
a) providing a peripheral blood composition comprising a population of lymphocytes from a human donor allogeneic to the recipient, said composition comprising T cells, in which the T cells are enriched for T cells reactive to neo-antigens in the recipient and
b) depleting of T cells reactive to antigens on non-cancerous tissues of the recipient,
to thereby generate a population of non-alloreactive T cells depleted of alloreactive T cells.

2. The method of claim 1, wherein the T cell is CD3⁺ T cell.

3. The method of claim 1, wherein neo-antigens are identified by whole exome sequencing and RNAseq of both cancerous and non-cancerous tissue of the same individual, and HLA binding algorithms applied to determine which neo-antigens bind HLA molecules shared by the donor and recipient.

4. The method of claim 1, wherein the donor has been immunized against one or more neo-antigen of the recipient.

5. The method of claim 4, wherein the immunization consists of intramuscular injection of antigen emulsified in an adjuvant or DNA vaccination plus electroporation.

6. The method of claim 1, wherein the T cells from an unvaccinated donor are stimulated ex vivo with one or more neo-antigen, with or without cytokines.

7. The method of claim 6, wherein the one or more neo-antigen used for stimulation is not encoded in the transcriptome or the whole exome of non-cancerous tissue of the same patient.

8. The method of claim 1, wherein the alloreactive T cells are selectively depleted by physical or chemical treatment, or wherein the non-alloreactive T cells are selected for infusion.

9. An allogeneic lymphocyte composition for administration to a human obtained by the method of claim 1.

10. An allogeneic lymphocyte composition comprising:
a population of T cells reactive to one or more tumor neo-antigens in the recipient, the frequency of such T cells being increased compared to their frequency in a tumor-free donor that has not been vaccinated with one or more neo-antigen.

11. The composition of claim 10, wherein the donor is vaccination with the one or more neo-antigen, with or without natural killer cells and other cells of the peripheral blood.

12. The composition of claim 10, wherein the T cell is CD3⁺ T cell.

13. A method of treating a cancer in a human, the method comprising;
(a) administering a lymphocyte composition made by the method of claim 1.

14. The method of claim 13, wherein the patient has a clinically, biochemically, or radiographically detectable neoplasm, or has a history of having a neoplasm.

15. The method of claim 13, wherein the lymphocyte composition is administered to a recipient who has undergone an allogeneic stem cell transplantation procedure from the same donor.

16. The method of claim 15, wherein the lymphocyte composition is the first infusion of cells from the donor.

17. The method of claim 16, wherein the lymphocyte composition is infused after treating the recipient with lymphodepleting, but non-myeloablative chemotherapy.

18. The method of claim 16, wherein the lymphocyte composition is infused into a tumor-bearing recipient treated with drugs to augment expression of the at least one antigen recognized by the infused cells.

19. The method of claim 16, wherein the lymphocyte composition is infused into a tumor-bearing recipient in combination with an immunological checkpoint inhibitor.

20. The method of claim 19, wherein the immunological checkpoint inhibitor is selected from ipilimumab, nivolumab, or pembrolizumab.

21. The method of claim 13, wherein the lymphocyte composition is infused into a recipient, said recipient having been treated with agents to reduce myeloid-derived suppressor cells or regulatory T cells.

22. The method of claim 13, wherein the lymphocyte composition is infused into a recipient with a neoplasm treated by a local ablative technique.

23. The method of claim 22, wherein the local ablative technique is selected from cryoablation, radiofrequency ablation, high intensity focused ultrasound, irreversible electroporation, external beam radiation, brachytherapy, or chemical destruction.

24. The method of claim 13, wherein the lymphocyte composition is infused with one or more populations of T cells specific for the same neo-antigens or sequential infused with T cells specific for different neo-antigens.

25. A method of making an allogeneic lymphocyte composition for treating cancer, the method comprising:
a) providing a peripheral blood composition from a human donor allogeneic to the recipient, the composition comprising T cells, wherein the T cells are enriched for T cells reactive to antigens expressed by the cancer and by the dispensible non-cancerous tissue of the recipient, but not by tissues of the donor, or by other tissues of the recipient with the exception of blood when this infusion is accompanied or preceded by an allogeneic bone marrow transplant from the same donor, and
b) depleting of T cells reactive to antigens on non-cancerous tissues of the recipient.

26. The method of claim 25, wherein the T cell is CD3⁺ T cell.

27. The method of claim 25, wherein the dispensible non-cancerous tissue is a sex organ.

28. A method of making an allogeneic lymphocyte composition for treating cancer, the method comprising:
a) providing a peripheral blood composition from a human donor allogeneic to the recipient, the composition comprising T cells, in which the T cells are enriched for T cells reactive to antigens expressed by the cancer and by the indispensable corresponding non-cancerous tissue of the recipient, but not by tissues of the donor, including the corresponding tissue of the donor that can be transplanted into the recipient, or by other tissues of the recipient, and
b) depleting of T cells reactive to antigens on non-cancerous tissues of the recipient.

29. The method of claim 28, wherein the T cell is CD3⁺ T cell.
